(19) United States
(12) Patent Application Publication
(10) Pub. No.: US 2025/0068698 A1
(43) Pub. Date: Feb. 27, 2025

(54) LEARNING DEVICE, STRESS ESTIMATION DEVICE, LEARNING METHOD, STRESS ESTIMATION METHOD, AND STORAGE MEDIUM

(71) Applicant: NEC Corporation, Minato-ku, Tokyo (JP)

(72) Inventors: Masanori TSUJIKAWA, Tokyo (JP); Tasuku KITADE, Tokyo (JP)

(73) Assignee: NEC Corporation, Minato-ku, Tokyo (JP)

(21) Appl. No.: 18/718,901

(22) PCT Filed: Dec. 23, 2021
(86) PCT No.: PCT/JP2021/047902

§ 371 Date: Jun. 12, 2024

(51) Int. Cl. G06F 18/241; G06N 20/00

(52) U.S. Cl. CPC G06F 18/241; G06N 20/00

(57) ABSTRACT

A learning device 1X mainly includes a classifying means 14X and a learning means 17X. The classification means 14X classifies observed feature values of subjects so that an index representing a correlation between the observed feature values and a stress value becomes higher than the correlation before the classification, wherein the stress value is a correct answer corresponding to the observed feature values. The learning means 17X trains, based on the observed feature values and the stress value which is the correct answer, stress estimation models for respective classes divided at least by the classification, wherein the stress estimation models each estimates a relation between the observed feature values and the stress value.

### TECHNICAL FIELD

The present disclosure relates to the technical fields of a learning device, a stress estimation device, a learning method, a stress estimation method, and a storage medium that perform processing on estimation of a stress state.

### BACKGROUND

There are known devices or systems for determining a stress state of a subject based on data measured from the subject. For example, Patent Literature 1 discloses a portable stress measurement device which determines a temporary stress degree of a target subject of estimation on each day based on biological data of the subject of estimation.

### CITATION LIST

### Patent Literature

Patent Literature 1: JP 2007-275287A

### SUMMARY

### Problem to be Solved

When estimating the stress level of the subject from the biological data of the subject, there is an issue that the estimation accuracy for unknown data is not stable.

In view of the above-described issues, it is an object of the present disclosure to provide a learning device, a stress estimation device, a learning method, a stress estimation method, and a storage medium configured to obtain stress estimation results of stable estimation accuracy.

### Means for Solving the Problem

In one aspect of the learning device, there is provided a learning device including:
a classification means configured to classify observed feature values of subjects so that an index representing a correlation between the observed feature values and a stress value becomes higher than the correlation before the classification,
   wherein the stress value is a correct answer corresponding to the observed feature
values; and a learning means configured to train, based on the observed feature values and the stress value which is the correct answer, stress estimation models for respective classes divided at least by the classification,
   wherein the stress estimation models each estimates a relation between the observed feature values and the stress value.

In one aspect of the stress estimation device, there is provided a stress estimation device including:
a classification score calculation means configured to calculate classification scores representing confidence levels in which observed feature values of an estimation subject, who is a target of stress estimation, belong to respective classes;
a stress estimation means configured to acquire stress values of the estimation subject estimated based on the observed feature values by stress estimation models corresponding to the respective classes; and
an integration means configured to calculate a stress estimate value obtained by integrating the stress values of the estimation subject estimated by the stress estimation models.

In one aspect of the learning method, there is provided a learning method executed by a computer, the learning method including:
classifying observed feature values of subjects so that an index representing a correlation between the observed feature values and a stress value becomes higher than the correlation before the classification,
   wherein the stress value is a correct answer corresponding to the observed feature values; and
training, based on the observed feature values and the stress value which is the correct answer, stress estimation models for respective classes divided at least by the classification,
   wherein the stress estimation models each estimates a relation between the observed feature values and the stress value.

The term “computer” herein include any electronic equipment (including a processor included in the electronic equipment) and may be configured by a plurality of electronic equipment.

In one aspect of the stress estimation method, there is provided a stress estimation method including:
calculating classification scores representing confidence levels in which observed feature values of an estimation subject, who is a target of stress estimation, belong to respective classes;
acquiring stress values of the estimation subject estimated based on the observed feature values by stress estimation models corresponding to the respective classes; and
calculating a stress estimate value obtained by integrating the stress values of the estimation subject estimated by the stress estimation models.

In one aspect of the storage medium, there is provided a storage medium storing a program executed by a computer, the program causing the computer to:
classify observed feature values of subjects so that an index representing a correlation between the observed feature values and a stress value becomes higher than the correlation before the classification,
   wherein the stress value is a correct answer corresponding to the observed feature values; and
train, based on the observed feature values and the stress value which is the correct answer, stress estimation models for respective classes divided at least by the classification,
   wherein the stress estimation models each estimates a relation between the observed feature values and the stress value.

In one aspect of the storage medium, there is provided a storage medium storing a program executed by a computer, the program causing the computer to:
calculate classification scores representing confidence levels in which observed feature values of an estimation subject, who is a target of stress estimation, belong to respective classes;
acquire stress values of the estimation subject estimated based on the observed feature values by stress estimation models corresponding to the respective classes; and
calculate a stress estimate value obtained by integrating the stress values of the estimation subject estimated by the stress estimation models.

### Effect

An example advantage according to the present invention is to estimate stress with a stable estimation accuracy, or, to train a stress estimation model to achieve such stress estimation.

### BRIEF DESCRIPTION OF THE DRAWINGS

FIG. 1 It illustrates a schematic configuration of a stress estimation system according to a first example embodiment.

FIG. 2 It illustrates an example of a hardware configuration of a stress estimation device common to each example embodiment.

FIG. 3 It illustrates an example of functional blocks in the learning phase of the information processing device according to the first example embodiment.

FIG. 4 It is a functional block diagram of a classification label generation unit and the classification model learning unit.

FIG. 5A is a diagram showing a processing overview of the first step of the classification label generation process.

FIG. 5B illustrates the second step of the classification label generation process.

FIG. 6 It illustrates an example of applying the first step and the second step to classes after subdivision.

FIG. 7 It illustrates an example of functional blocks of a certain feature selection unit.

FIG. 8 It illustrates a histogram which aggregates correlations for a certain type of observed feature values.

FIG. 9 It illustrates an example of a flowchart indicating a procedure of a learning process that is executed by an information processing device in the learning phase according to the first example embodiment.

FIG. 10 It is an example of functional blocks in the estimation phase executed by the information processing device according to the first example embodiment.

FIG. 11 It illustrates an example of a flowchart indicating a procedure of a stress estimation process that is executed by the information processing device in the estimation phase according to the first example embodiment.

FIG. 12 It illustrates a schematic configuration of a stress estimation system in the second example embodiment.

FIG. 13 It is a block diagram of a learning device in a third example embodiment.

FIG. 14 It illustrates an example of a flowchart executed by the learning device in the third example embodiment.

### EXAMPLE EMBODIMENTS

Hereinafter, example embodiments of a learning device, a stress estimation device, a learning method, a stress estimation method, and a storage medium will be described with reference to the drawings.

### First Example Embodiment

### (1) System Configuration

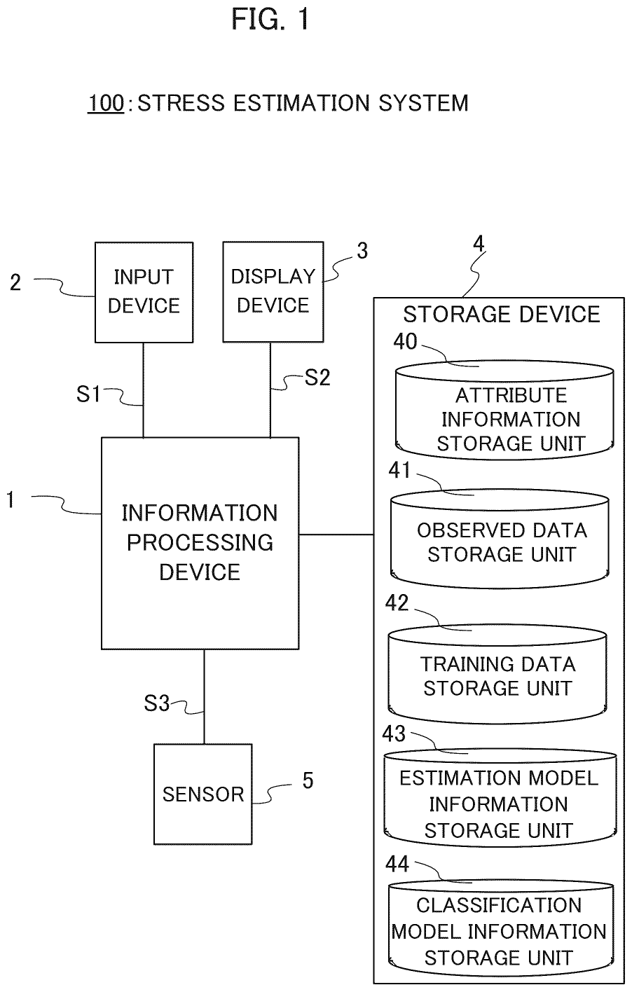

FIG. 1 shows a schematic configuration of a stress estimation system 100 according to the first example embodiment. The stress estimation system 100 trains models (also referred to as “stress estimation models”) configured to estimate the stress of a person, and performs stress estimation based on the trained stress estimation models. Hereafter, a person who is a target of stress estimation is referred to as “estimation subject”, and a person who is a target of measurement in the generation of training data (learning sample) necessary to train the stress estimation models is also referred to as “sample subject”. In addition, when the estimation subject and the sample subject are not distinguished in particular, these are simply referred to as “subject”. The term “estimation subject” may indicate a sports player or an employee subject to management of the stress state by the organization, or may indicate an individual user.

The stress estimation system 100 mainly includes an information processing device 1, an input device 2, a display device 3, a storage device 4, and a sensor 5.

The information processing device 1 performs data communication with the input device 2, the display device 3, and the sensor 5 through a communication network or through direct communication through wireless or wired communication. Based on the input signal “S1” supplied from the input device 2 and the sensor signal “S3” supplied from the sensor 5, the information processing device 1 collects information required for training the stress estimation 10 model or for estimating the stress of the estimation subject using the stress estimation models, and stores the collected information in the storage device 4. Further, the information processing device 1 generates the display signal “S2” based on the estimated result of the stress state (specifically, the stress value representing the degree of stress) of the estimation subject, and supplies the generated display signal S2 to the display device 3. It is noted that the stress estimated by the information processing device 1 in the present example embodiment is assumed to be a chronic stress which is a stress in a long term (chronic) in units of several days, a week or a month.

The input device 2 is one or more interfaces configured to receive user input (manual input) of information regarding each estimation subject. The user who inputs information using the input device 2 may be the estimation subject itself or may be a person who manages or supervises the activity of the estimation subject. Examples of the input device 2 include a variety of user input interfaces such as a touch panel, a button, a keyboard, a mouse, and a voice input device. The input device 2 supplies the input signal S1 generated based on the input from the user to the information processing device 1. The display device 3 displays information based on the display signal S2 supplied from the information processing device 1. Examples of the display device 3 include a display and a projector.

The sensor 5 measures a biological signal or the like of the estimation subject and supplies the measured biological signal or the like to the information processing device 1 as a sensor signal S3. In this instance, the sensor signal S3 may be any biological signal (including vital information) such as heartbeat, EEG, amount of perspiration, amount of hormonal secretion, cerebral blood flow, blood pressure, body temperature, electromyogram, electrocardiogram, respiration rate, pulse wave, acceleration regarding estimation subject. The sensor 5 may be a device that analyzes blood of the estimation subject and outputs the analysis result as a sensor signal S3. Examples of the sensor 5 may include a wearable terminal worn by the estimation subject, a camera for photographing the estimation subject, a microphone for generating a voice signal of the estimation subject's utterance, one or more sensors mounted on a terminal, such as a personal computer and a smartphone, operable by an estimation subject. Examples of the above-described wearable terminal include a GNSS (global navigation satellite system) receiver, an accelerometer, and any other sensor that detects a biological signal, and the wearable terminal outputs an output signal from the respective sensors as a sensor signal S3. The sensor 5 may supply information corresponding to the operation quantity of the personal computer or the smart phone to the information processing device 1 as the sensor signal S3. The sensor 5 may also output a sensor signal S3 representing biological data (including sleep time) from the subject during sleep of the subject. The sensor signal S3 is used to generate feature values (also referred to as “observed feature values”) representing the observed features of an observed subject.

The storage device 4 is a memory configured to store various information necessary for estimating the stress state. The storage device 4 may be an external storage device, such as a hard disk, connected to or embedded in the information processing device 1, or may be a storage medium such as a flash memory. The storage device 4 may be a server device that performs data communication with the information processing device 1. Further, the storage device 4 may be configured by a plurality of devices.

The storage device 4 functionally includes an attribute information storage unit 40, an observed data storage unit 41, a training data storage unit 42, an estimation model information storage unit 43, and a classification model information storage unit 44.

The attribute information storage unit 40 stores the attribute information relating to the attribute of the subject. Here, the term “attribute” herein includes, for example, the personality of a subject, the stress tolerance, the gender, the job type, the age, the perception tendency, and a combination thereof. The attribute information may be generated by the information processing device 1 and stored in the storage device 4, or may be generated in advance by a device other than the information processing device 1 and stored in the storage device 4. The attribute information may include information generated based on the results of the questionnaire answered by the subject. Examples of the questionnaire to measure the personality of a subject include the Big Five personality test. The attribute information is associated with the identification information regarding the subject and is stored in the attribute information storage unit 40.

The observed data storage unit 41 stores observed data generated based on the sensor signal S3 or the like which the information processing device 1 acquires from the sensor 5. In the present example embodiment, for example, the observed data is information in which observed feature values are associated with observation time information, activity information indicating the activity state of the subject at the time of the observation, and identification information regarding the subject, wherein examples of the activity state include the intensity of physical exercise, the intensity of mental activity such as a mental workload, a state of sitting, walking, or running, a state of awaking or sleeping. For convenience of explanation, it is assumed that the observed data storage unit 41 stores the observed data regarding an estimation subject, and the training data storage unit 42 stores the observed data regarding sample subjects.

The observed feature values are index values indicating the features of data observed form the subject or a vector (feature vector) which includes the index values as elements. Examples of the observed feature values include feature values based on a biological feature such as amount of perspiration, acceleration, skin temperature, and pulse wave, and feature values based on a behavioral feature regarding an activity (action) of the subject such as an amount of operating a device. Here, the process of converting the sensor signal S3 into the observed feature values may be executed by the information processing device 1, or may be executed by a device other than the information processing device 1. In this case, the observed feature values may be generated from the sensor signal S3 based on any method of calculating feature values from a biological signal or any other method. The activity information is generated by the information processing device 1 or any other device based on, for example, position information, acceleration, or the like included in the sensor signal S3.

The training data storage unit 42 stores training data used for training the stress estimation model. The training data is data generated for a plurality of sample subjects, and includes a plurality of sets of observed data of a sample subject and a correct answer stress value (stress data) based on answers of questionnaires by the sample subject. For example, the correct answer stress value is a PSS (Perceived Stress Scale) value. The PSS value is calculated from the response to a PSS questionnaire for measuring the dynamical stress that changes over time. Further, as will be described later, in the learning stage, the information processing device 1 generates the classification labels indicating the classes of the observed feature values for respective sample subjects based on the attributes of the respective sample subjects, and stores the generated classification labels in the training data storage unit 42.

The estimation model information storage unit 43 stores parameters (in other words, parameters necessary for configuring the stress estimation model) of the stress estimation models trained by the information processing device 1. The stress estimation models are models each of which estimates the relation between observed feature values of a subject and the stress value of the subject. Each stress estimation model is trained to output the stress value of a target subject of estimation when a combination (a feature vector) of specific types of observed feature values of the target subject is inputted thereto. Here, the stress estimation model may be any machine learning model (including a statistical model) such as a neural network, a support vector machine, and the like.

In addition, as will be described later, multiple stress estimation models are trained for respective classes by using the training data divided for respective classes classified to increase the correlation between the observed data and the stress values which are the correct answers. In this case, each stress estimation model may have a suitable architecture for each class. Hereafter, the term “class” shall mean a class (group) uniquely associated with a target stress estimation model of training. It is noted that the same number of classes is provided as the number of the target stress estimation models of training. The estimation model information storage unit 43 stores the information of the parameters necessary for configuring these stress estimation models. For example, when a stress estimation model is a model based on a neural network such as a convolutional neural network, the estimation model information storage unit 43 stores information of parameters such as the layer structure, the neuron structure of each layer, the number of filters and the filter size in each layer, and the weight for each element of each filter.

The classification model information storage unit 44 stores parameters (in other words, parameters necessary for configuring the classification model) of the classification model trained by the information processing device 1. Here, the classification model is a model configured to estimate the relation between one or more attributes of a subject and the class into which the observed feature values of the subject should be classified. The classification model is trained so as to output a score (also referred to as “classification score”) representing the confidence level for each candidate class when the attribute information regarding the subject is inputted thereto. The architecture of a learning model of such a classification model is a model based on a neural network such as a convolutional neural network, for example. The higher the confidence level for a given class is, the higher the classification score for that class becomes. The classification model is trained on the basis of the attribute information of the sample subjects and the classification labels representing the classes of the observed feature values of the respective sample subjects.

The configuration of the stress estimation system 100 shown in FIG. 1 is an example, and various changes may be made to the configuration. For example, the input device 2 and the display device 3 may be configured integrally. In this case, the input device 2 and the display device 3 may be configured as a tablet-type terminal that is integral or separate from the information processing device 1. In this case, the information processing device 1, the input device 2, the display device 3, and the sensor 5 (which may include the storage device 4) may be configured as a smartphone or a wearable terminal used by the subject. Further, the information processing device 1 may be configured by a plurality of devices. In this case, the plurality of devices constituting the information processing device 1 performs transmission and reception of information necessary for executing preassigned processing among the plurality of devices. In this case, the information processing device 1 functions as an information processing system.

### (2) Hardware Configuration of Information Processing Devise

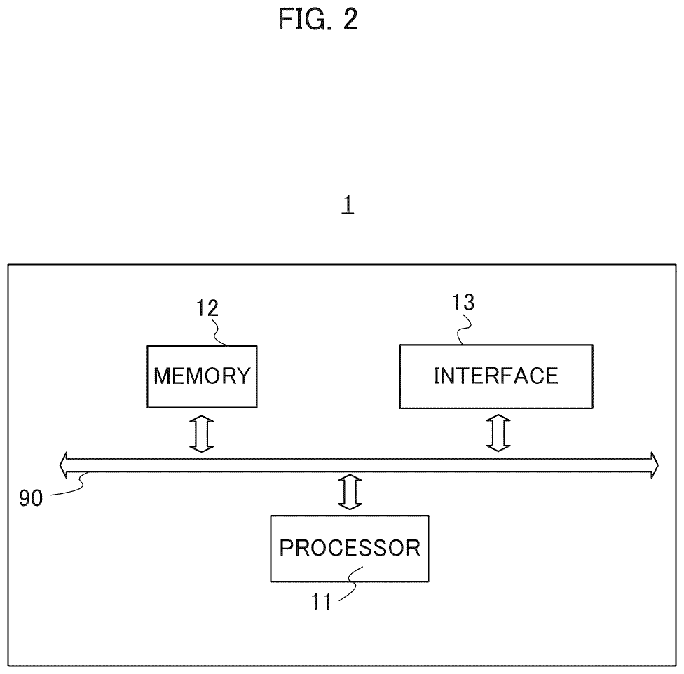

FIG. 2 shows a hardware configuration of the information processing device 1. The information processing device 1 includes a processor 11, a memory 12, and an interface 13 as hardware. The processor 11, memory 12 and interface 13 are connected to one another via a data bus 90.

The processor 11 functions as a controller (arithmetic unit) configured to control the entire information processing unit 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 comprises a variety of volatile and non-volatile memories, such as a RAM (Random Access Memory), a ROM (Read Only Memory), and a flash memory. Further, a program for executing a process executed by the information processing device 1 is stored in the memory 12. A part of the information stored in the memory 12 may be stored by one or more external storage devices that can communicate with the information processing device 1, or may be stored by a storage medium detachable from the information processing device 1.

The interface 13 is one or more interfaces for electrically connecting the information processing device 1 to other devices. Examples of these interfaces include a wireless interface, such as a network adapter, for transmitting and receiving data to and from other devices wirelessly, and a hardware interface, such as a cable, for connecting to other devices.

The hardware configuration of the information processing device 1 is not limited to the configuration shown in FIG. 2. For example, the information processing device 1 may include at least one of the input device 2 and the display device 3. Further, the information processing device 1 may be connected to or incorporate a sound output device such as a speaker.

### (3) Learning Phase

Next, the process in the learning phase executed by the information processing device 1 will be described. In summary, the information processing device 1 classifies the observed feature values so that the correlation between the observed feature values and the correct answer a stress value becomes high, and trains the stress estimation models for respective classified classes. Thus, the information processing device 1 trains stress estimation models specialized for respective classes classified according to the bias in the stress tendency, and acquires stress estimation models capable of performing stress estimation with high accuracy for unknown data which is not used for training.

### (3-1) Functional Blocks

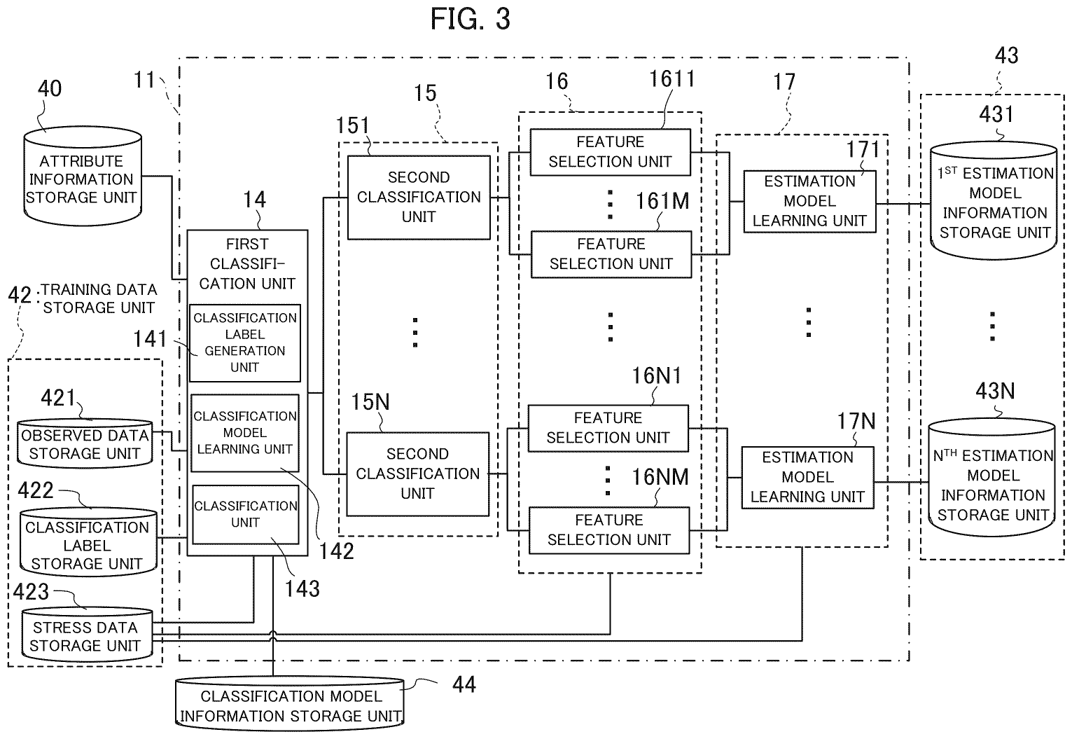

FIG. 3 is an example of functional blocks in the learning phase of the information processing device 1. In the learning phase, the processor 11 of the information processing device 1 functionally includes a first classification unit 14, “N” (N is an integer of 2 or more) second classification units 15 (151 to 15N), “M” (M is an integer of 2 or more) feature selection units 16 (1611 to 16NM), and N estimation model learning units 17 (171 to 17N). The training data storage unit 42 functionally includes an observed data storage unit 421, a classification label storage unit 422, and a stress data storage unit 423. Furthermore, the estimation model information storage unit 43 functionally includes a first estimation model information storage unit 431 to an N-th estimation model information storage unit 43N for storing parameters of N target stress estimation models of learning, respectively. In FIG. 3, blocks to exchange data with each other are connected to each other by a solid line, but the combination of blocks to exchange data with each other is not limited to the combination shown in the drawings. The same applies to the drawings of other functional blocks described below.

The first classification unit 14 executes processing related to the first classification in which the observed feature values used for learning are classified (clustered) into N classes to increase the correlation between the observed feature values and the correct answer stress values. The first classification unit 14 functionally includes a classification label generation unit 141, a classification model learning unit 142, and a classification unit 143.

The classification label generation unit 141 refers to the attribute information storage unit 40, the observed data storage unit 421, and the stress data storage unit 423, and generates a classification label corresponding to each sample subject. As described below, in the present example embodiment, in the generation process of the classification label described below, the classification label generation unit 141 adaptively determines the number “N” indicating the number of classes (that is, the number of stress estimation models) of the classification labels. The classification label generation unit 141 stores the generated classification labels in the classification label storage unit 422. Details of the processing of the classification label generation unit 141 will be described later.

The classification model learning unit 142 trains the classification model by referring to the attribute information storage unit 40 and the classification label storage unit 422. In this case, for example, the classification model learning unit 142 extracts a set of the attribute information and the classification label corresponding to each sample subject in order, and updates the parameters of the classification model. In this case, the parameters of the classification model are determined so that the error (loss) between the classification result outputted by the classification model when the attribute information is inputted thereto and the correct answer class indicated by the classification label is minimized. Examples of the attribute information inputted to the classification model include: an index value indicating a personality, gender, job type, race, age, height, weight, amount of muscle, life habit, or exercise habit; and a combination of these index values (vector value). The algorithm for determining parameters to minimize the error may also be any learning algorithm used in machine learning, such as the gradient descent method and the error back propagation method. The classification model learning unit 142 stores the parameters of the trained classification model in the classification model information storage unit 44.

The classification unit 143 extracts the observed feature values for learning from the observed data storage unit 421, and classifies (makes clusters of) the extracted observed feature values into N classes according to the classification labels stored in the classification label storage unit 422. Thus, the classification unit 143 prepares N sets of observed feature values that are biased in terms of stress or biological features or the like. Then, the classification unit 143 supplies the observed feature values for respective classes to the corresponding second classification units 151 to 15N according to the classes.

Instead of classifying the observed feature values for learning into N classes according to the classification labels stored in the classification label storage unit 422, the classification unit 143 may classify the observed feature values for learning into N classes based on the classification result by the classification model trained by the classification model learning unit 142. In this case, the classification unit 143 extracts the attribute information associated with a sample subject from the attribute information storage unit 40 and classifies the observed feature values of the sample subject as a class having the highest classification score that is outputted by the classification model when the extracted attribute information is inputted to the classification model. Accordingly, since the first classification using the classification model is performed in the same manner as in the estimation phase described below even in the learning phase, the estimation accuracy in the estimation phase is expected to be improved.

The second classification units 15 (151 to 15N) performs the second classification to classify a set of observed features for each class supplied from the first classification unit 14 into M sub-classes based on the observation target of the observed feature values or the activity state of the subject at the time of observation. Thus, the second classification unit 15 further divides the observed feature values to be handled differently in the stress estimation. Then, each of the second classification units 151 to 15N supplies the sets of the observed feature values divided into M sub-classes based on the second classification to the feature selection units 16 (1611 to 16NM), respectively.

Here, the “observation target” is the observation target of the raw data used to calculate the observed feature values, and examples of the observation target include perspiration, acceleration, skin temperature, pulse wave, and any other biological features. Accordingly, for example, if the observed feature values are based on biological features, the “classification based on observation target” indicates classification of the observed feature values such that observed feature values relating to perspiration, observed feature values relating to acceleration, observed feature values relating to skin temperature, and observed feature values relating to pulse wave are classified as different sub-classes, respectively. The “classification based on activity state” is, for example, a classification into sub-classes according to the levels (e.g., stationary state, walking state, running state) of the exercise intensity at the time of observation of the subject. The information indicating the observation target and the activity state corresponding to each observed feature value is stored in association with the each observed feature value in the observed data storage unit 421, for example.

The feature selection units 16 (1611 to 16NM) select, based on correlations with correct answer stress values, observed feature values (referred to as “stress estimation feature values”) to be inputted into the stress estimation models, from N×M sets of observed feature values classified on the basis of the first classification and the second classification, respectively. It is herein assumed that each feature values selection unit 16 selects, as the stress estimation feature values, R (R is an integer of 0 or more) types of observed feature values. Details of the process by the feature selection unit 16 will be described later. The number of the feature selection units 16 may not be necessarily M which is uniform for each class, and an appropriate number of the feature selection units 16 may be provided for each class. Similarly, the value of R may be different among the feature selection units 16.

Based on the stress estimation feature values selected by the feature values selection units 16 and the correct answer stress values from the stress data storage unit 423, the estimation model learning units 17 (171 to 17N) train the stress estimation models prepared for respective classes classified based on the first classification. In this case, each estimation model learning unit 17 acquires plural sets of input data and correct answer data, wherein M×R pieces of stress estimation feature values supplied from the M feature values selection units 16 are used as the input data and the corresponding stress values acquired from the stress data storage unit 423 are used as the correct answer data. Then, each estimation model learning unit 17 trains the corresponding stress estimation model on the basis of the above-described plural sets of the input data and the correct answer data.

In the learning of the stress estimation model, for example, each estimation model learning unit 17 extracts the above-described sets of input data and correct data in order, and updates the parameters of the corresponding stress estimation model. In this case, each estimation model learning unit 17 determines the parameters of the corresponding stress estimation model so that the error (loss) between the estimated result outputted by the corresponding stress estimation model when the input data is inputted thereto and the stress value (PSS value in this case) which is correct answer data is minimized. The algorithm for determining parameters to minimize the error may be any learning algorithm used in machine learning, such as the gradient descent method and the error back propagation method. The estimation model learning units 17 store the parameters of the trained stress estimation models in the first estimation model information storage unit 431 to the Nth estimation model information storage unit 43N, respectively.

Each component of the first classification unit 14, the second classification unit 15, the feature selection unit 16, and the estimation model learning unit 17 described in FIG. 3 can be realized by the processor 11 executing a program, for example. Additionally, the necessary programs may be recorded on any non-volatile storage medium and installed as necessary to realize each component. It should be noted that at least a portion of each of these components may be implemented by any combination of hardware, firmware, and software, without being limited to being implemented by software based on a program. At least some of these components may also be implemented using user programmable integrated circuit such as, for example, a FPGA (Field-Programmable Gate Array) and a microcontroller. In this case, the integrated circuit may be used to realize a program functioning as each of the above components. Further, at least a part of the components may be configured by ASSP (Application Specific Standard Produce), ASIC (Application Specific Integrated Circuit) or a quantum processor (quantum computer control chip). Thus, each of the above-described components may be realized by various hardware. Furthermore, each of these components may be implemented by cooperation of a plurality of computers, for example, using cloud computing technology.

### (3-2) Details of Classification Label Generation Unit

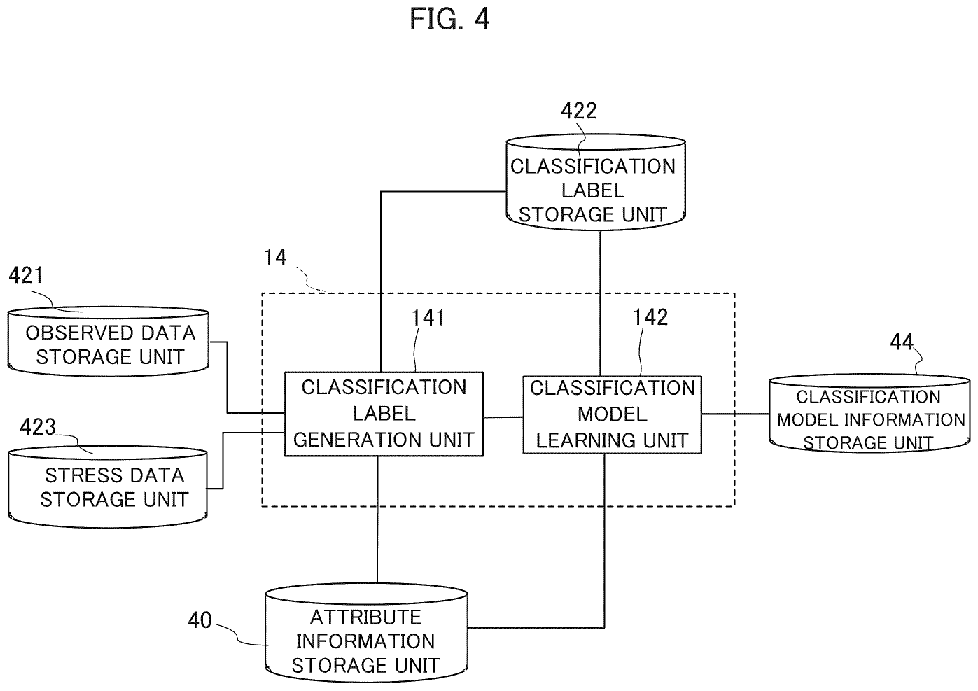

FIG. 4 is a detailed functional block diagram of the classification label generation unit 141 and the classification model learning unit 142 included in the first classification unit 14. Based on the attribute information stored in the attribute information storage unit 40, the observed feature values stored in the observed data storage unit 421, and the stress values that are correct answers stored in the stress data storage unit 423, the classification label generation unit 141 generates the classification labels, and stores the generated classification labels in the classification label storage unit 422. The classification model learning unit 142 trains the classification model on the basis of the classification labels stored in the classification label storage unit 422 and the attribute information stored in the attribute information storage unit 40, and stores parameters of the classification model obtained by training in the classification model information storage unit 44.

The classification label generation unit 141 classifies the observed feature values for each sample subject into a plurality of classes based on the attribute information. Then, the classification label generation unit 141 randomly shuffles (transfers) the observed feature values among the classes, and adopts the shuffle if the correlation between the observed feature values for each class and their correct answer a stress value becomes higher than the correlation before shuffling. Then, the classification label generation unit 141 performs classification of the observed feature values by repeating the shuffling to increase the correlation between the observed feature values for each class and the correct answer stress values, and generates the classification labels representing the classification result.

Here, a specific example of a method of generating the classification label that is executed by the classification label generation unit 141 will be described. In this specific example, as the first step, provisional class subdivision based on the attribute information is performed, and as the second step, the above-described shuffling among the subdivided classes is performed. Then, the classification label generation unit 141 repeatedly executes the first step and the second step until it is determined that class subdivision is unnecessary. Here, as an example, the classification label generation unit 141 performs class subdivision hierarchically by repeating division into halves. The division number (number of partitions) in the case of hierarchical class subdivision may be 3 or more.

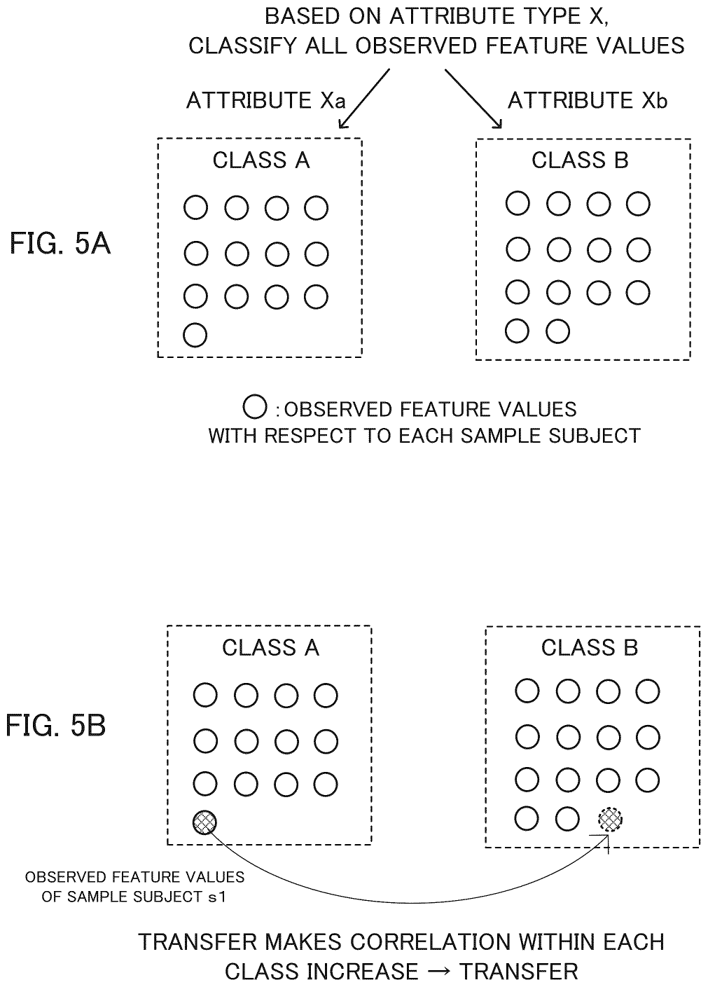

FIG. 5A illustrates a processing outline of the first step of the classification label generation process. Here, a set of observed features for each sample subject is indicated by a circle.

First, the classification label generation unit 141 classifies all the observed feature values for learning into two classes (class A and class B) based on the attribute type “X”. Here, the classification label generation unit 141 tentatively classifies the observed feature values of such sample subjects whose attribute type X is an attribute Xa into the class A and tentatively classifies the observed feature values of such sample subjects whose attribute type X is the attribute Xb into the class B. For example, the attribute type X is personality, gender, job type, race, age, height, weight, muscle mass, habit, exercise habit, and each of attributes Xa and Xb is a category or a range of index values in the attribute type X. For example, if attribute type X is gender, the attribute Xa is male, and the attribute Xb is female.

Then, the classification label generation unit 141 determines that subdivision of the class is necessary and proceeds to the second step if the correlation (also referred to as “observation-stress correlation”) between the observed feature values and the correct answer stress values increases by the provisional classification based on the attribute type X in the first step. Specifically, first, the classification label generation unit 141 calculates an observation-stress correlation based on the entire observed feature values before classification into the class A and the class B, an observation-stress correlation based on the observed feature values classified into the class A, and an observation-stress correlation based on the observed feature values classified into the class B. Then, when the observation-stress correlation regarding the class A and the observation-stress correlation regarding the class B are both higher than the observation-stress correlation before the classification, the classification label generation unit 141 determines that subdivision into the class A and the class B is necessary, and performs the second step for the class A and the class B. The classification label generation unit 141 may determine that subdivision is necessary if either one of the observation-stress correlation regarding the class A or the observation-stress correlation regarding the class B is higher than the overall observation-stress correlation before the classification. In another example, the classification label generation unit 141 may determine that subdivision is necessary if the average of the observation-stress correlation regarding the class A and the observation-stress correlation regarding the class B is higher than the overall observation-stress correlation before the classification.

Here, a supplementary description will be given of the “correlation” calculated in the first step. The classification label generation unit 141 may calculate the correlation coefficient as the correlation, or may calculate the index value representing any other correlation such as the mutual information content. Further, the classification label generation unit 141 may perform an arbitrary normalization process for eliminating an influence caused by a difference in the number of samples in the calculation of the index value described above.

FIG. 5B illustrates an outline of the second step for the class A and the class B. The classification label generation unit 141 tentatively classifies the observed feature values into the classes A and B based on the attribute information in the first step, and then, in the second step, shuffles the observed feature values for each sample subject so that the observation-stress correlation is improved. In FIG. 5B, the classification label generation unit 141 calculates the observation-stress correlation of the class A and the observation-stress correlation of the class B on the assumption that the observed feature values of the sample subject s1, which are provisionally classified into the class A based on the attribute information, are tentatively transferred to the class B. Then, if both of the observation-stress correlation of the class A and the observation-stress correlation of the class B increases due to the transfer, the classification label generation unit 141 adopts the transfer of the observed features of the sample subject s1 from the class A to class B. Then, the classification label generation unit 141 determines whether or not the transfer is required and performs the transfer if needed, for the observed feature values of all the sample subjects included in the class A and the class B. Thus, the classification label generation unit 141 can classify the observed feature values into the class A and the class B to increase the observation-stress correlation.

In some embodiments, upon determining that the observation-stress correlation of the class from which the observed feature values of the sample subject s1 transfer decreases due to the transfer and the observation-stress correlation of the class to which the observed feature values of the sample subject s1 transfer increases, the classification label generation unit 141 may cause the observed feature values of the sample subject s1 to exist in both the class A and the class B. In this instance, the classification label generation unit 141 classifies the observed features of the sampled subject s1 into both of the classes A and B. This allows the classification label generation unit 141 to improve the observation-stress correlation of both of the class A and the class B.

In some embodiments, if the observation-stress correlation of the class from which the observed feature values of the sample subject s1 transfer increases due to the transfer and the observation-stress correlation of the class from which the observed feature values of the sample subject s1 transfer decreases due to the transfer, the classification label generation unit 141 does not need to classify the observed feature values of the sample subject s1 into neither the class A nor the class B. In other words, the observed features of the sampled subject s1 are not used for training. This also allows the classification label generation unit 141 to improve the observation-stress correlations of both of the class A and the class B.

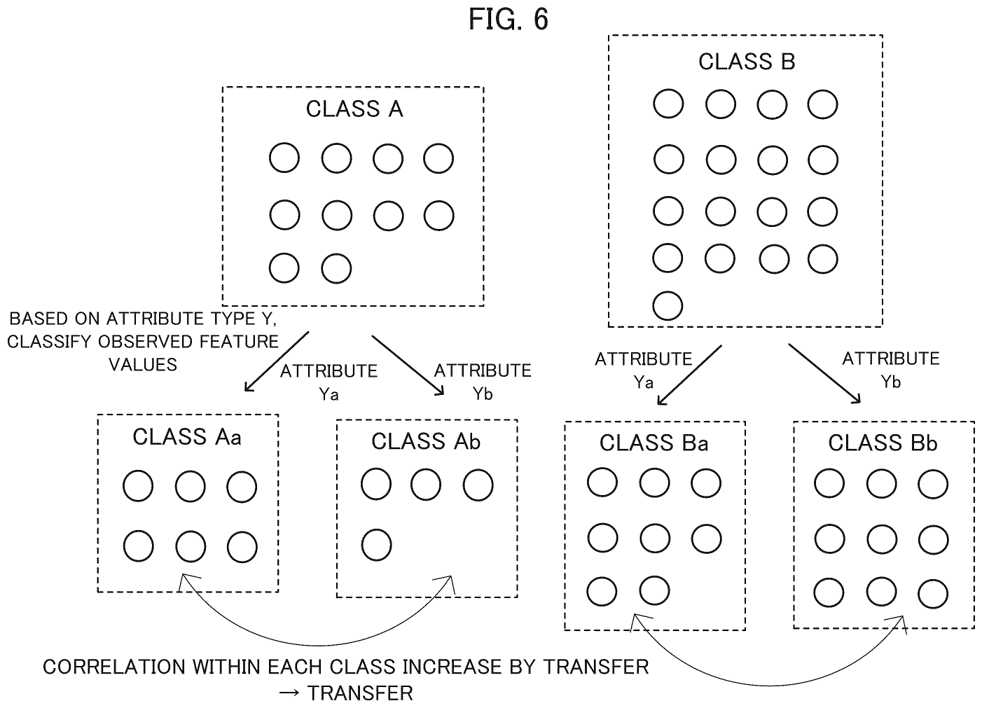

After execution of the second step, the classification label generation unit 141 performs the first step for the class A and the class B, respectively, and performs the second step for the classes subdivided at the first step. FIG. 6 shows an example of applying the first step and the second step to the class A and the class B, respectively.

Here, as an example, at the first step, the classification label generation unit 141 subdivides each of the classes A and B into two classes, based on the attribute type “Y”. Specifically, the classification label generation unit 141 classifies the observed feature values of the class A that corresponds to the attribute “Ya” into the class Aa, and classifies the observed feature values of the class A that corresponds to the attribute “Yb” into the class Ab. In addition, the classification label generation unit 141 classifies the observed feature values of the class B corresponding to the attribute Ya into the class Ba and classifies the observed feature values of the class B corresponding to the attribute Yb into the class Bb.

Instead of classifying based on an attribute type Y different from the attribute type X used at the first step, the classification label generation unit 141 may perform classification based on the attribute type X used at the first step. In this case, for example, the classification label generation unit 141 classifies the observed feature values of the class A into the class Aa and the class Ab based on the attribute “Xaa” and the attribute “Xab” into which the attribute Xa is classified (categorized) in more detail, and classifies the observed feature values of the class B into the class Ba and the class Bb based on the attribute “Xba” and the attribute “Xbb” into which the attribute Xb is subdivided.

Then, upon determining that the observation-stress correlation increases by the provisional classification based on the attribute information at the first step, the classification label generation unit 141 shuffles, based on the second step, the observed feature values for each sample subject among the subdivided classes. In the example shown in FIG. 6, since the observation-stress correlation is increased by subdividing the class A into the class Aa and the class Ab, the classification label generation unit 141 shuffles the observed features between the class Aa and the class Ab so that the observation-stress correlation is improved. Similarly, since the correlation is increased by subdividing the class B into the class Ba and the class Bb, the classification label generation unit 141 shuffles the observed features between the class Ba and the class Bb so as to improve the observation-stress correlation.

As such, the classification label generation unit 141 hierarchically increases the number of classes by applying the first step and the second step to each generated class. Then, the classification label generation unit 141 ends the process when the observation-stress correlation does not increase due to the class subdivision in any class. Then, the classification label generation unit 141 generates the classification labels indicating the classes to which the observed feature values for respective sample subjects belong at the processing end, and stores the generated classification labels in the classification label storage unit 422.

As described above, the classification label generation unit 141 hierarchically subdivides the classes, and then determines the observed feature values belonging to each class based on the variation in the observation-stress correlation due to the transfer of the observed feature values among the classes. Then, the classification label generation unit 141 performs the subdivision of classes such that the observation-stress correlation increases by the subdivision among the current classes until there is no class whose observation-stress correlation increases by the subdivision. Thus, the classification label generation unit 141 can adaptively determine the number N of classes and generate the classification labels so that the observation-stress correlation increases.

Instead of determining whether or not to subdivide the class based on the provisional classification result based on the attribute information, the classification label generation unit 141 may determine whether or not to subdivide the class after the completion of the shuffling at the second step. For example, in the example shown in FIG. 6, the classification label generation unit 141 determines that the class Aa and the class Ab should be provided if the observed-stress correlation of the class Aa and the observed-stress correlation of the class Ab after the second step are higher than the observed-stress correlation of the entire class A. On the other hand, the classification label generation unit 141 determines that subdivision of the class A into the class Aa and the class Ab is unsuitable if the observation-stress correlation of the class Aa and the observation-stress correlation of the class Ab after the second step execution are not higher than the observation-stress correlation of the entire class A. Therefore, in this case, the classification label generation unit 141 generates the classification labels which define the classes of the observed feature values tentatively classified into the class Aa and the class Ab as the class A. According to this example, it is possible to more accurately determine whether or not subdivision of the class is necessary.

The classification label generation unit 141 may set the number N of classes to a fixed value instead of adaptively determining the number N of classes. In this case, the classification label generation unit 141 performs processing corresponding to the second step after classifying the observed feature values for respective sample subjects into N classes based on the attribute information to thereby determine the observed feature values belonging to the respective N classes. In this case, at the second step, the classification label generation unit 141 may transfer a set of the observed feature values for a sample subject to the class in which the increase in the observation-stress correlation is the largest. If the observation-stress correlation of any class to which the observed feature values transfer does not increase, the classification label generation unit 141 may not change the class of the observed feature values nor classify the observed feature values into any class (not used for training).

In yet another example, the classification label generation unit 141 may set a plurality of candidates (candidate class numbers) for the number N of classes and determine the number N of classes to be a candidate class number having the highest observation-stress correlation. In this case, the classification label generation unit 141 performs classification based on the first step and the second step by setting each candidate class number as the number N of classes, and then determines the number N of classes to be a candidate class number with which the observation-stress correlation for each class after the classification is the highest among candidate class numbers. For example, when candidate class numbers are “2”, “3”, and “4”, respectively, the classification label generation unit 141 compares the average of the observation-stress correlation of each class after classification when the number of classes is fixed to two, the average of the observation-stress correlation of each class after classification when the number of classes is fixed to three, and the average of the observation-stress correlation of each class after classification when the number of classes is fixed to four. Then, the classification label generation unit 141 determines the number N of classes to be a candidate class number having the highest average of the observed-stress correlations of all classes, and generates the classification labels based on the classification result when the number N of classes is set to the candidate class number. Also in this example, the classification label generation unit 141 can determine the number N of classes and the classification labels such that the observation-stress correlation is the highest.

### (3-3) Details of Feature Selection Unit

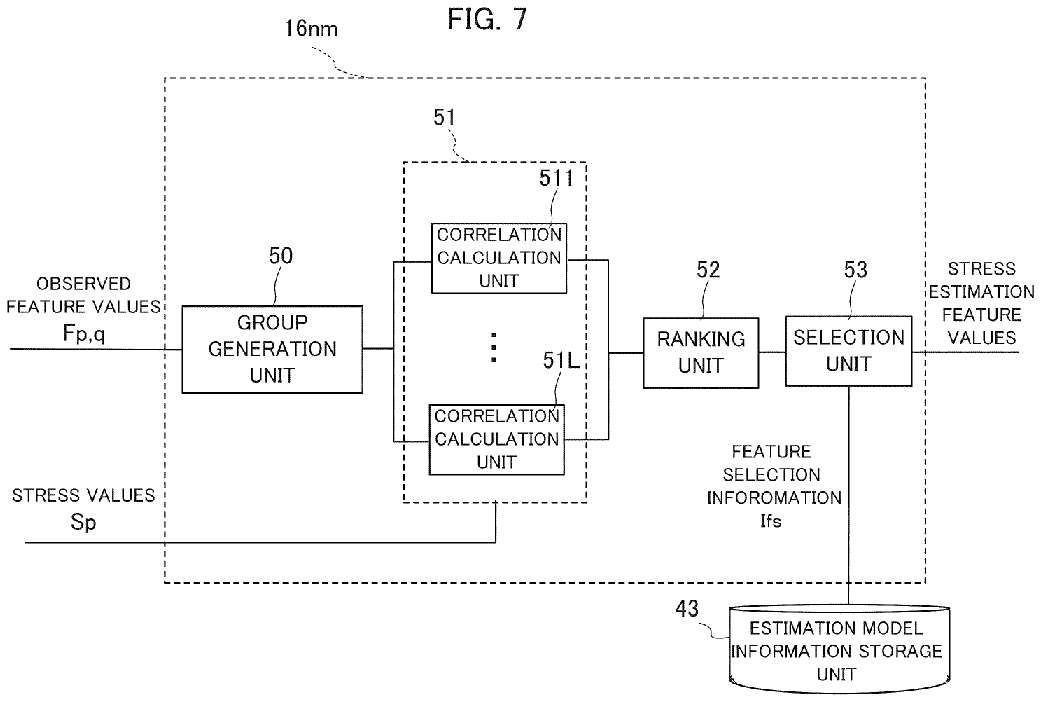

Next, a detailed description of the process that the feature selection units 16 (1611 to 16NM) execute. FIG. 7 illustrates an example of functional blocks of a certain feature values selection unit 16nm (“n”, “m” each is an integer satisfying 1≤n≤N, 1≤m≤M). The feature values selection unit 16nm functionally includes a group generation unit 50, a correlation calculation unit 51, a ranking unit 52, and a selection unit 53.

The feature selection unit 16nm acquires the observed feature values “F_{p,q}” from the second classification unit 15n and acquires a correct stress value (PSS value) “Sₚ” corresponding to the observed feature values F_{p,q} from the stress data storage unit 423. Here, “p” indicates the index of the sample subject (1≤p≤P, P is an integer of 2 or more), and “q” indicates the index of the type of observed feature values (1≤q≤Q, Q is an integer satisfying “Q≥R”). It is noted that there are generally a large number of types of observed feature values (e.g., several tens of thousands), and examples of the types include, in the case of the feature values relating to perspiration, the maximum value of the perspiration, the minimum value of the perspiration, the median value of the perspiration, the average value of the perspiration, and any other various indices relating to perspiration such as any other statistics.

The group generation unit 50 randomly extracts a predetermined number of the observed feature values F_{p,q} for L (L is an integer of 1 or more) times, and thereby generates L groups, each of which includes the predetermined number of the extracted observed feature values F_{p,q}. In this case, for example, if there are 100 sample subjects corresponding to the observed feature values F_{p,q} (i.e., P=100), the group generation unit 50 randomly extracts L times as the observed feature values F_{p,q} corresponding to 50 sample subjects and forms a group of the observed feature values F_{p,q} of the 50 sample subjects extracted in every trial. Then, the group generation unit 50 supplies each group of the observed feature values F_{p,q} to the correlation calculation units 511 to 51L, respectively.

Based on the groups of the observed feature values F_{p,q} supplied from the group generation unit 50, the correlation calculation units 51 (511 to 51L) calculate correlations (correlation coefficients) between the observed feature values F_{p,q} and the stress values Sₚ for each type q of the observed feature values F_{p,q}. As the correlation coefficients, any one of Pearson's product-moment correlation coefficient, Spearman's rank correlation coefficient, Kendol's rank correlation coefficient, or the combination thereof (such as average value thereof) may be used. In other words, the correlation calculation unit 51 calculates the correlation between the observed feature values F_{p,q} and the stress value Sₚ with respect to each group generated by the group generation unit 50 and with respect to each type q of the observed feature values F_{p,q}.

The ranking unit 52 ranks each type q of the observed feature values F_{p,q}, based on the calculation results by the L correlation calculation units 511 to 51L. In this instance, the ranking unit 52 calculates a score (also referred to as “correlation score”) for each type q of the observed feature values F_{p,q}, based on the calculation results by the L correlation calculation units 511 to 51L, and determines that the higher the correlation score is, the higher the ranking becomes. In this case, the ranking unit 52 calculates the correlation score based on the statistical value such as the average of the correlations among the groups and the sign inversion degree to be described later. The method of calculating the correlation score will be described later.

The selection unit 53 selects the observed feature values F_{p,q} corresponding to the top R types in the rankings determined by the ranking unit 52 as the stress estimation feature values. In this instance, the selection unit 53 stores information (also referred to as “feature selection information Ifs”) indicating the types of the observed feature values selected as the stress estimation feature values in the estimation model information storage unit 43. As described later, the feature selection information Ifs is used in the process of selecting the stress estimation feature values to be inputted into the stress estimation model from the observed feature values in the estimation phase.

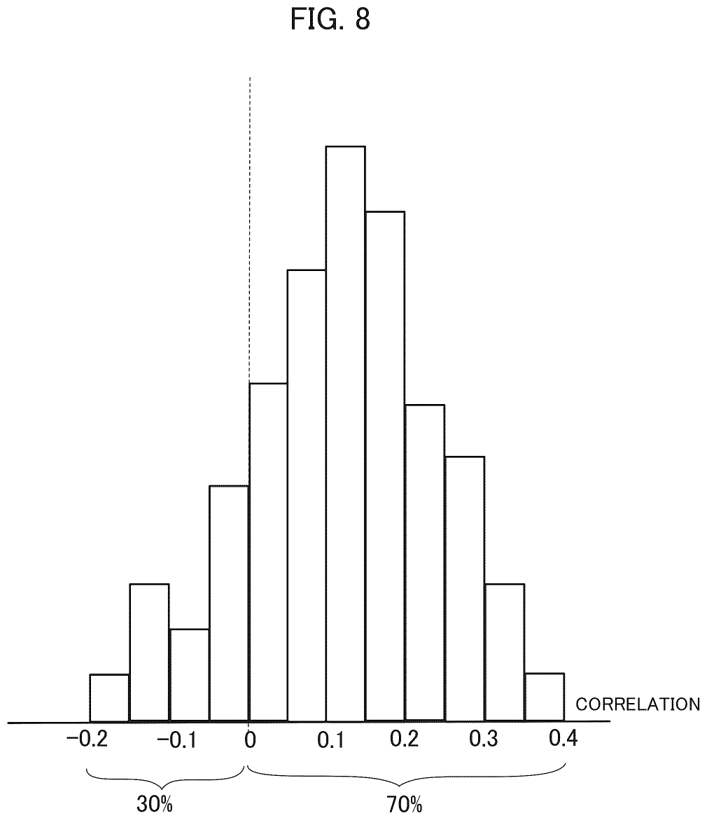

Here, a specific example of a method of calculating the correlation score by the ranking unit 52 will be described. FIG. 8 shows a histogram in which the correlations for a target type q of calculation of the correlation score are aggregated based on the calculation results by the correlation calculation units 511 to 51L. Although the histogram is shown here for convenience of explanation, the generation of the histogram is not an essential process in the calculation of the correlation score.

In this case, first, the correlation calculation unit 51 calculates the average (0.15 in this case) of the correlations (correlation coefficients) calculated by the correlation calculation units 511 to 51L for the target type q based on the computation results by the correlation calculation units 511 to 51L. In addition, the correlation calculation unit 51 calculates, as the sign inversion degree, the proportion of the minority signs in the case where positive and negative signs of the calculated correlations are aggregated. In the example shown in FIG. 8, since the positive signs are the majority, the correlation calculation unit 51 recognizes the proportion (0.3) of the negative signs as the sign inversion degree. The sign inversion degree ranges from 0 to 0.5. Then, for example, the correlation calculation unit 51 determines the correlation score for the target type q to be a value obtained by multiplying the absolute value of the average of the correlations by the value (which ranges from 0.5 to 1) obtained by subtracting the sign inversion degree from 1 as a weight, as follows:

Correlation Score=|Average of Correlation|×(1−Sign Inversion Degree).

In the example shown in FIG. 8, the correlation score of the target type q is 0.105 (=|0.15|× 0.7).

The calculation method of the correlation score is not limited to the above-described expression, and an arbitrary expression or a look-up table, in which the correlation score is defined to have a positive correlation with the average of the correlations and to have a negative correlation with the sign inversion degree, may be used.

Such a functional configuration of the feature values selection unit 16nm as shown in FIG. 7 allows for suitable selection of the stress estimation feature values to be observed feature values correlated with the stress values regardless of the individual difference.

### (3-4) Processing Flow

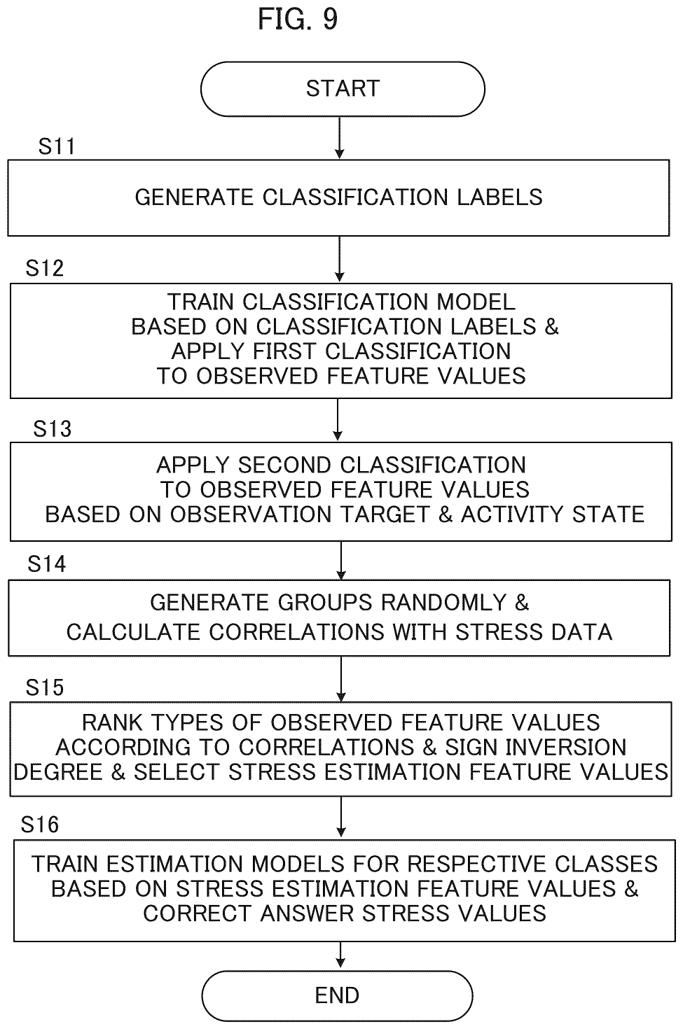

FIG. 9 is an example of a flowchart illustrating a procedure of a learning process that is executed by the information processing device 1 in a learning phase according to the first example embodiment.

First, the first classification unit 14 of the information processing device 1 performs a process of generating the classification labels (step S11). In this case, the information processing device 1 determines the number N of classes and generates the classification labels based on the processing described in the section “(3-2) Details of Classification Label Generation Unit”.

Next, the first classification unit 14 trains the classification model based on the classification labels and performs the first classification of the observed feature values for training stored in the observed data storage unit 421 (step S12). In this case, the information processing device 1 may execute the first classification based on the classification labels, or may execute the first classification based on the trained classification model and the attribute information stored in the attribute information storage unit 40.

Next, the second classification unit 15 of the information processing device 1 performs the second classification of the observed feature values according to the observation target of the observed feature values and the corresponding activity states at the time of observation of sample subjects (step S13). In this case, for example, based on the second classification according to the type of the observed biological features and the exercise intensity of each sample subject, the second classification unit 15 further classifies the observed features into M sub-classes for each of the sets of observed features divided into N classes.

Next, for each set of the observed feature values divided into N×M sub-classes, the feature values selection unit 16 of the information processing device 1 generates groups by random, and then calculates correlations of each generated group with the corresponding correct answer stress values included in the training data for each type of the observed feature values (step S14). Furthermore, the feature values selection unit 16 ranks the types of the observed feature values according to the correlations and the sign inversion degree for each set of the observed feature values in divided sub-class units, and selects the observed feature values corresponding to the types of the top R types of observed feature values as the stress estimation feature values (step S15).

Then, based on the stress estimation feature values and the corresponding correct answer stress values included in the training data, the estimation model learning unit 17 of the information processing device 1 trains the stress estimation models for respective classes divided according to the first classification (step S16). The information processing device 1 outputs the feature selection information Ifs related to the stress estimation feature values selected at step S15 and the parameters of the stress estimation models trained at step S16 as the learning result. Specifically, the information processing device 1 stores the feature selection information Ifs and the parameters of the stress estimation models in the storage device 4. Thus, the information processing device 1 can store information in the storage device 4 needed in the estimation phase.

### (4) Estimation Phase

Next, the process in the estimation phase executed by the information processing device 1 will be described. The information processing device 1 estimates the stress value of an estimation subject based on the classification model and the stress estimation model that are trained in the learning phase.

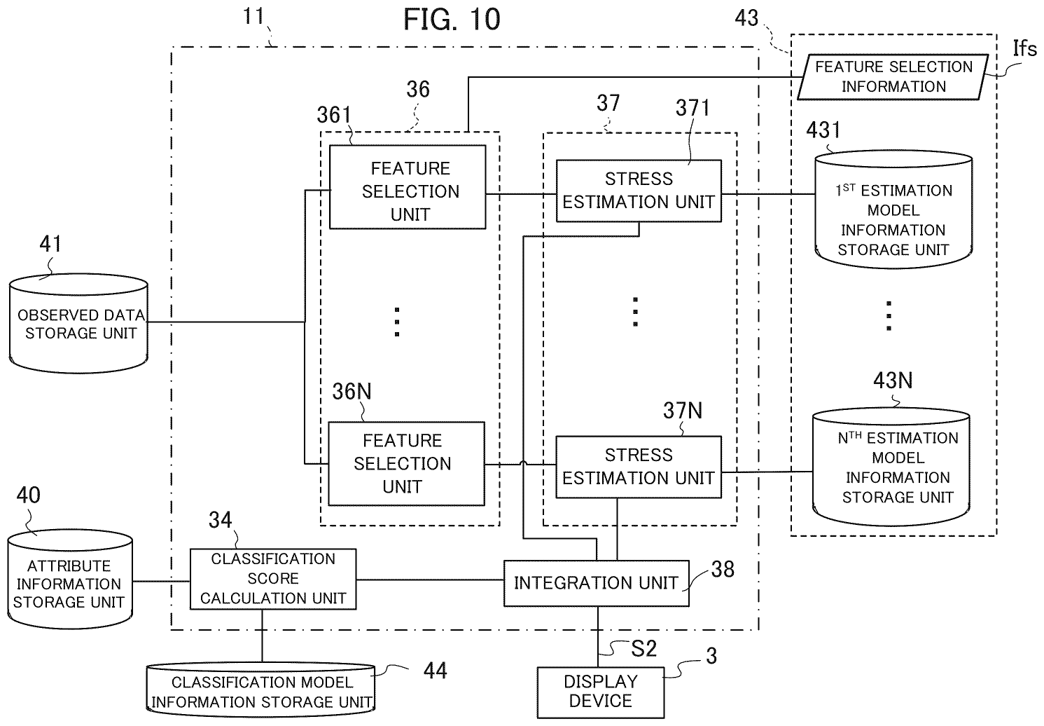

FIG. 10 illustrates an example of functional blocks in the estimation phase of the information processing device 1. In the estimation phase, the processor 11 of the information processing device 1 functionally includes a classification score calculation unit 34, N feature values selection units 36 (361 to 36N), N stress estimation units 37 (371 to 37N), and an integration unit 38. The first estimation model information storage unit 431 to the N-th estimation model information storage unit 43N included in the estimation model information storage unit 43 store the parameters of the N stress estimation models in which learning has already been performed in the learning phase.

Based on the extracted attribute information, the classification score calculation unit 34 extracts the attribute information regarding the estimation subject from the attribute information storage unit 40 and calculates the classification scores to respective classes (i.e., N classes corresponding to the first estimation model to the Nth estimation model) provided in the first classification of the learning phase. In this case, the classification score calculation unit 34 acquires the classification scores for respective classes outputted from the classification model by inputting the attribute information described above to the classification model based on the classification model information stored in the classification model information storage unit 44. Then, the classification score calculation unit 34 supplies the acquired classification scores for respective classes to the integration unit 38.

Based on the feature selection information Ifs stored in the estimation model information storage unit 43, the feature selection units 36 (361 to 36N) selects stress estimation feature values from the observed feature values of the estimation subject extracted from the observed data storage unit 41. In this instance, the feature selection unit 36n (where n is any integer from 1 to N) extracts, from the observed feature values of the estimation subject, the same types of observed feature values as the types of stress estimation feature values indicated by the feature selection information Ifs generated by the feature selection unit 16n1 to the feature selection unit 16nM, as the stress estimation feature values. Then, the feature values selection unit 36n supplies the extracted stress estimation feature values to the corresponding stress estimation unit 37n.

The stress estimation units 37 (371 to 37N) estimate the stress of the estimation subject based on the stress estimation models and stress estimation feature values supplied from the feature selection units 36 (361 to 36N). In this instance, the stress estimation unit 37n (where n is any integer from 1 to N) configures the corresponding n-th estimation model by referring to the corresponding n-th estimation model information storage unit 43n. The stress estimation unit 37n acquires the stress of the estimation subject outputted by the n-th estimation model by inputting the stress estimation feature values supplied from the corresponding feature selection unit 36n to the configured n-th estimation model. The stress value outputted by each stress estimation model corresponds to a candidate value for the stress value of the estimation subject that is finally estimated by the integration unit 38. Each stress estimation unit 37 (371 to 37N) supplies the stress of the estimation subject outputted by the corresponding stress estimation model to the integration unit 38.

The integration unit 38 integrates the stress values supplied from the stress estimation units 37 (371 to 37N) through weighting (i.e., performs weighted averaging) based on the classification scores for respective classes supplied from the classification score calculation unit 34. Then, the integration unit 38 outputs the integrated stress value as a finally-estimated value (also referred to as “stress estimate value”) of the stress of the estimation subject. For example, the integration unit 38 generates a display signal S2 for displaying the information regarding the integrated stress estimate value, and supplies the display device 3 with the display signal S2, thereby causing the display device 3 to display the information regarding the stress estimate value. In this case, the integration unit 38 can integrate the stress values of the estimation subject outputted by the stress estimation models by the weighting process based on the classification scores to thereby calculate the stress estimate value with high accuracy.

Instead of or in addition to performing control for displaying the stress estimate value itself, the integration unit 38 may perform control for displaying: information on the stress level that is determined based on the comparison between the stress estimate value and a predetermined threshold value; and/or information on the advice in accordance with the stress level. The viewer of the display device 3 in this case, for example, may be an estimation subject, or may be a person who manages or supervises the estimation subject. Further, the integration unit 38 may perform the audio output of the information regarding the stress estimate value by a sound output device (not shown).

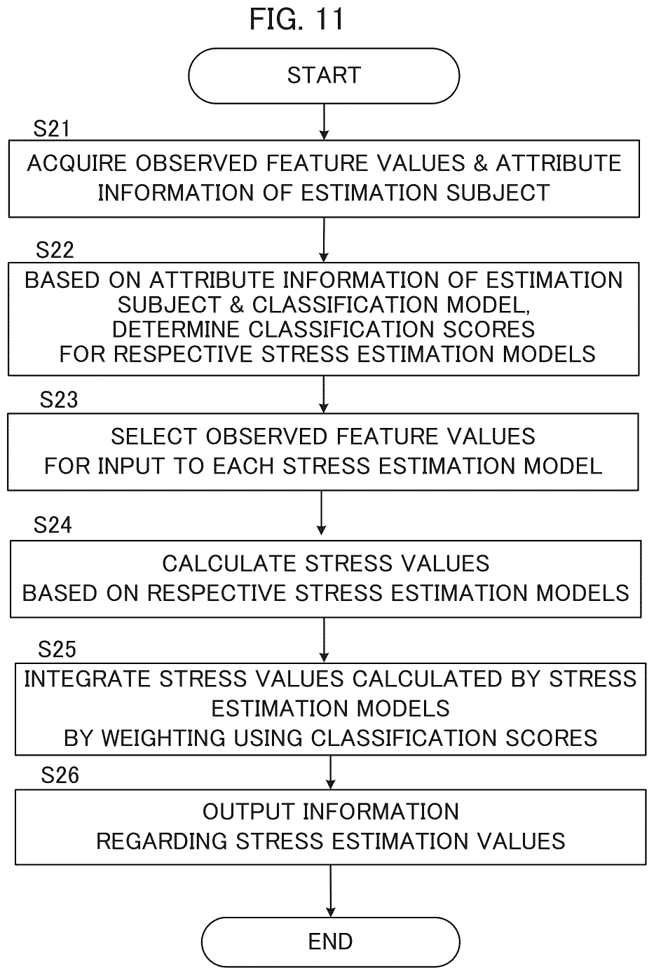

FIG. 11 is an example of a flowchart illustrating a procedure of a stress estimation process that is executed by the information processing device 1 in an estimation phase. The timing at which the stress estimation process is performed may be a timing requested by the user based on the input signal S1 or may be a predetermined timing.

First, the information processing device 1 acquires the observed feature values of the estimation subject and the attribute information regarding the estimation subject (step S21). In this case, for example, the information processing device 1 acquires the above-described observed feature values from the observed data storage unit 41 and acquires the above-described attribute information from the attribute information storage unit 40.

Next, the classification score calculation unit 34 of the information processing device 1 determines the classification scores corresponding to respective N stress estimation models based on the attribute information regarding the estimation subject and the classification model to which the parameters stored in the classification model information storage unit 44 are applied (step S22). In this case, the classification score calculation unit 34 acquires the classification scores of respective classes, which are uniquely corresponding to stress estimation models, from the classification model in which the attribute information is inputted.

Then, the feature values selection unit 36 of the information processing device 1 selects the observed feature values to be inputted respectively into the N stress estimation models provided for respective classes (step S23). In this instance, the feature values selection unit 36 refers to the corresponding feature selection information Ifs and selects the stress estimation feature values, which are the observed feature values to be inputted into the stress estimation model, from the observed feature values acquired at step S21. The processing order of the processes at step S22 and step S23 should be no particular order, and may be executed simultaneously or in parallel.

The stress estimation unit 37 of the information processing device 1 calculates the stress values of respective stress estimation models (that is, for respective classes), on the basis of the selected stress estimation feature values and respective stress estimation models that are configured based on the parameters stored in the estimation model information storage unit 43 (step S24). In this case, the stress estimation unit 37 calculates a stress value for each stress estimation model by inputting the stress estimation feature values supplied from the feature selection unit 36 to each stress estimation model configured by referring to the estimation model information storage unit 43.

The integration unit 38 of the information processing device 1 calculates the integrated stress estimate value by weighting the stress values outputted by the respective stress estimation models based on the classification scores for respective classes determined at step S22 (step S25). The integration unit 38 of the information processing device 1 outputs the information on the stress estimate value (step S26).

### (5) Modifications

Next, a description will be given of modifications applicable to the first example embodiment.

### First Modification

The stress estimation model may be provided for each sub-class classified according to both of the first classification and the second classification, instead of being provided for each class classified according to the first classification.

In this case, in the learning phase, the information processing device 1 provides stress estimation models corresponding to N×M present feature selection units 1611 to 16NM, respectively. Then, the information processing device 1 trains the stress estimation models using the stress estimation feature values outputted by the corresponding feature selection unit 16 as the input data and using the stress values indicated by the corresponding stress data as the correct answer data. In the estimation phase, similarly to the feature values selection units 16 in the learning phase, N×M pieces of the feature values selection units 36 are provided, and the stress estimation units 371 to 37N input the stress estimation feature values outputted by the corresponding M feature values selection units 36 into the corresponding M stress estimation models, respectively. Then, the integration unit 38 calculates the stress estimate value by performing the weighted averaging based on the stress values outputted by the NxM stress estimation models and the classification scores set for respective classes.

Thus, even in the present modification, the information processing device 1 can accurately estimate the stress state of the estimation subject from the observed feature values, that are not used for learning, based on the stress estimation models that are trained for respective sets biased in the stress tendency.

### Second Modification

The stress that the information processing device 1 estimates is not limited to a chronic stress, but may be the short-term stress that is a stress in a relatively short period (several minutes to about a day).

### Third Modification

In the learning phase, the information processing device 1 may perform learning of the stress estimation models without performing at least one of the second classification by the second classification unit 15 and/or the feature selection by the feature selection unit 16. Even in this case, the information processing device 1 can perform learning of the stress estimation models for respective classes set based on the first classification to increase the correlation between the observed feature values and the stress values, and acquire the stress estimation models capable of performing the stress estimation with high accuracy for the unknown data not used for training. When the feature selection by the feature selection unit 16 is not performed, the information processing device 1 also does not perform the feature selection by the feature selection unit 36 in the estimation phase.

### Second Example Embodiment

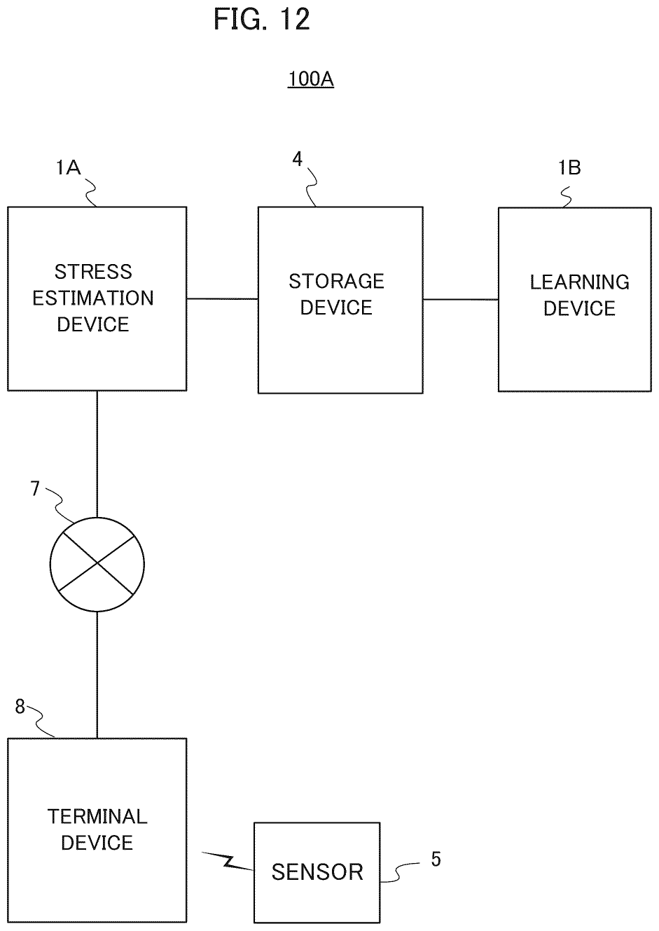

FIG. 12 shows a schematic configuration of a stress estimation 100A according to a second example embodiment. The stress estimation system 100A according to the second example embodiment includes a stress estimation device 1A which performs processing of the estimation phase executed by the information processing device 1 according to the first example embodiment, a learning device 1B which performs processing of the learning phase executed by the information processing device 1 according to the first example embodiment, a storage device 4, and a terminal device 8 and a sensor 5 which are used by the estimation subject. Hereinafter, the same components as those in the first example embodiment are appropriately denoted by the same reference numerals, and a description thereof will be omitted.

In the second example embodiment, the stress estimation device 1A functions as a server and the terminal device 8 functions as a client. The stress estimation device 1A and the terminal device 8 perform data communication via the network 7.

The learning device 1B is equipped with a hardware configuration identical to the hardware configuration of the information processing device 1 shown in FIG. 2, and the processor 11 of the learning device 1B is equipped with functional blocks shown in FIG. 3. The learning device 1B performs processes such as learning of the stress estimation model, learning of the classification model, and generation of the feature selection information Ifs, based on information stored in the storage device 4.

The terminal device 8 is a terminal used by a user who is an estimation subject. The terminal device 8 is equipped with an input function, display function, and a communication function, and functions as the input device 2 and the display device 3 shown in FIG. 1. The terminal device 8 may be, for example, a personal computer, a tablet-type terminal such as a smartphone, a PDA (Personal Digital Assistant), and the like. The terminal device 8 is electrically connected to the sensor 5, such as a wearable sensor worn by the user, and transmits the biological signal or the like (that is, information corresponding to the sensor signal S3 in FIG. 1) of the estimation subject outputted by the sensor 5 to the stress estimation device 1A through the network 7. Further, the terminal device 8 receives the user input or the like relating to the answer of a questionnaire, and transmits the information (information corresponding to the input signal S1 in FIG. 1) generated by the user input to the stress estimation device 1A. The sensor 5 may be incorporated in the terminal device 8. Further, the sensor 5 may be equipped with the functions of the terminal device 8 to perform data communication with the stress estimation device 1A.

The stress estimation device 1A is equipped with the same hardware configuration as the hardware configuration of the information processing device 1 shown in FIG. 2, and the processor 11 of the stress estimation device 1A is equipped with the functional blocks shown in FIG. 10. The stress estimation device 1A receives information corresponding to the input signal S1 and the sensor signal S3 in FIG. 1 from the terminal device 8 via the network 7, and stores the received information in the storage device 4. The stress estimation device 1A refers to the parameters of the stress estimation models, the parameters of the classification model, and the feature selection Ifs which are acquired by the learning device 1B, and executes a stress estimation process of the estimation subject. The stress estimation device 1A transmits an output signal for outputting the stress estimation result to the terminal device 8 through the network 7 in response to the display request from the terminal device 8.

Thus, in some embodiments, the stress estimation system 100A in the second example embodiment conducts the learning phase and the estimation phase separately by different devices, to thereby train the stress estimation models and estimate the stress using the stress estimation models in the same manner as in the first example embodiment. Further, in the second example embodiment, the stress estimation device 1A estimates the stress condition of the estimation subject based on the biological signal or the like of the estimation subject received from the terminal used by the estimation subject, and can suitably present the estimation result to the estimation subject on the terminal.

### Third Example Embodiment

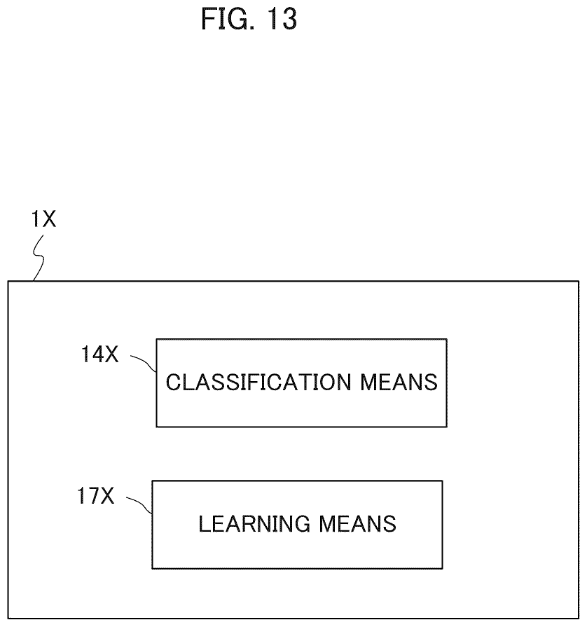

FIG. 13 is a block diagram of a learning device 1X according to the third example embodiment. The learning device 1X mainly includes a classification means 14X and a learning means 17X. The learning device 1X may be configured by a plurality of devices.

The classification means 14X is configured to classify observed feature values of subjects so that an index representing a correlation between the observed feature values and a stress value becomes higher than the correlation before the classification, wherein the stress value is a correct answer corresponding to the observed feature values. Examples of the classification means 14X include a first classification unit 14 in the first example embodiment (including modifications, hereinafter the same) and the second example embodiment.

The learning means 17X is configured to train, based on the observed feature values and the stress value which is the correct answer, stress estimation models for respective classes divided at least by the classification, wherein the stress estimation models each estimates a relation between the observed feature values and the stress value. In this case, the same number of stress estimation models are provided as the number of classes and the stress estimation models are trained per class. Examples of the learning means 17X include the estimation model learning unit 17 according to the first example embodiment and the second example embodiment.

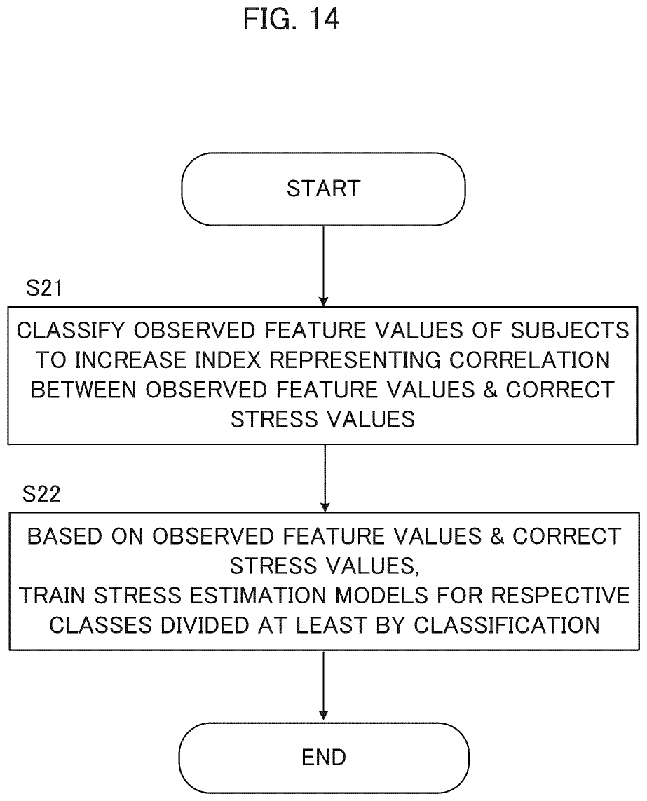

FIG. 14 is an example of the flowchart that is executed by the learning device 1X in the third example embodiment. First, the classification means 14X classifies observed feature values of subjects so that an index representing a correlation between the observed feature values and a stress value becomes higher than the correlation before the classification, wherein the stress value is a correct answer corresponding to the observed feature values (step S31). The learning means 17X trains, based on the observed feature values and the stress value which is the correct answer, stress estimation models for respective classes divided at least by the classification, wherein the stress estimation models each estimates a relation between the observed feature values and the stress value (step S32).

According to the third example embodiment, the learning device 1X can train the stress estimation models for respective groups having biases in the stress tendency to acquire the stress estimation models capable of performing the stress estimation with high accuracy.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### Supplementary Note 1

A learning device comprising:
a classification means configured to classify observed feature values of subjects so that an index representing a correlation between the observed feature values and a stress value becomes higher than the correlation before the classification,
   wherein the stress value is a correct answer corresponding to the observed feature values; and
a learning means configured to train, based on the observed feature values and the stress value which is the correct answer, stress estimation models for respective classes divided at least by the classification,
   wherein the stress estimation models each estimates a relation between the observed feature values and the stress value.

### Supplementary Note 2

The learning device according to Supplementary Note 1, further comprising
a classification model learning means configured to train, based on attribute information indicating attributes of the subjects and a result of the classification, a classification model configured to estimate a relation between the attribute and the class.

### Supplementary Note 3

The learning device according to Supplementary Note 2,
wherein the classification means is configured to classify, based on the attribute information and the classification model, the observed feature values for training the respective stress estimation models.

### Supplementary Note 4

The learning device according to any one of Supplementary Notes 1 to 3,
wherein the classification means is configured to classify, based on attribute information indicating attributes of the subjects, the observed feature values into the classes and then shuffle the observed feature values among the classes to increase the index for each of the classes.

### Supplementary Note 5

The learning device according to any one of Supplementary Notes 1 to 4,
wherein the classification means is configured to classify the observed feature values into classes and then further subdivide a class, among the classes, in which the index is increased by subdividing the class.

### Supplementary Note 6

The learning device according to any one of Supplementary Notes 1 to 5, further comprising:
a second classification means configured to classify the observed feature values based on at least one of an observation target of the observed feature values and/or activity states of the subjects; and
a feature selection means configured to select stress estimation feature values, which are feature values for stress estimation, from the observed feature values classified based on the classification by the classification means and the classification by the second classification means,
wherein the learning means is configured to train the stress estimation models for the respective classes, based on the stress estimation feature values and stress values which are correct answer corresponding to the stress estimation feature values.

### Supplementary Note 7

The learning device according to Supplementary Note 6,
wherein the feature selection means is configured to select the stress estimation feature values based on a correlation between the observed feature values classified based on the classification by the classification means and the classification by the second classification means and the stress values.

### Supplementary Note 8

A stress estimation device comprising:
a classification score calculation means configured to calculate classification scores representing confidence levels in which observed feature values of an estimation subject, who is a target of stress estimation, belong to respective classes;
a stress estimation means configured to acquire stress values of the estimation subject estimated based on the observed feature values by stress estimation models corresponding to the respective classes; and
an integration means configured to calculate a stress estimate value obtained by integrating the stress values of the estimation subject estimated by the stress estimation models.

### Supplementary Note 9

The stress estimation device according to Supplementary Note 8,
wherein the classification score calculation means is configured to calculate the classification scores based on classification model and attribute information indicating an attribute of the estimation subject, and
wherein the classification model is a model configured to classify observed feature values for training so that an index representing a correlation between the observed feature values and a stress value becomes higher than the correlation before the classification,
wherein the stress value is a correct answer corresponding to the observed feature values.

### Supplementary Note 10

The stress estimation device according to Supplementary Note 8 or 9, further comprising
a feature selection means configured to select stress estimation feature values, which are feature values for stress estimation, from the observed feature values,
wherein the stress estimation means is configured to calculate the stress estimate value obtained by integrating stress values of the estimation subject,
   the stress values being estimated based on the stress estimation feature values by stress estimation models corresponding to the respective classes.

### Supplementary Note 11

A learning method executed by a computer, the learning method comprising:
classifying observed feature values of subjects so that an index representing a correlation between the observed feature values and a stress value becomes higher than the correlation before the classification,
   wherein the stress value is a correct answer corresponding to the observed feature values; and
training, based on the observed feature values and the stress value which is the correct answer, stress estimation models for respective classes divided at least by the classification,
   wherein the stress estimation models each estimates a relation between the observed feature values and the stress value.

### Supplementary Note 12

A stress estimation method executed by a computer, the stress estimation method comprising:
calculating classification scores representing confidence levels in which observed feature values of an estimation subject, who is a target of stress estimation, belong to respective classes;
acquiring stress values of the estimation subject estimated based on the observed feature values by stress estimation models corresponding to the respective classes; and
calculating a stress estimate value obtained by integrating the stress values of the estimation subject estimated by the stress estimation models.

### Supplementary Note 13

A storage medium storing a program executed by a computer, the program causing the computer to:
classify observed feature values of subjects so that an index representing a correlation between the observed feature values and a stress value becomes higher than the correlation before the classification,
   wherein the stress value is a correct answer corresponding to the observed feature values; and
train, based on the observed feature values and the stress value which is the correct answer which are the correct answer, stress estimation models for respective classes divided at least by the classification,
   wherein the stress estimation models each estimates a relation between the observed feature values and the stress value.

### Supplementary Note 14

A storage medium storing a program executed by a computer, the program causing the computer to:
calculate classification scores representing confidence levels in which observed feature values of an estimation subject, who is a target of stress estimation, belong to respective classes;
acquire stress values of the estimation subject estimated based on the observed feature values by stress estimation models corresponding to the respective classes; and
calculate a stress estimate value obtained by integrating the stress values of the estimation subject estimated by the stress estimation models.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1 Information processing device
1A Stress estimation device
1B, 1X Learning device
2 Input device
3 Display device
4 Storage device
5 Sensor
8 Terminal device
100,100A Stress estimation system

What is claimed is:

1. A learning device comprising:
at least one memory configured to store instructions; and
at least one processor configured to execute the instructions to:
classify observed feature values of subjects so that an index representing a correlation between the observed feature values and a stress value becomes higher than the correlation before the classification,
wherein the stress value is a correct answer corresponding to the observed feature values; and
train, based on the observed feature values and the stress value which is the correct answer, stress estimation models for respective classes divided at least by the classification,
wherein the stress estimation models each estimate a relation between the observed feature values and the stress value.

2. The learning device according to claim 1,
wherein the at least one processor is configured to further execute the instructions to train, based on attribute information indicating attributes of the subjects and a result of the classification, a classification model configured to estimate a relation between the attribute and the class.

3. The learning device according to claim 2,
wherein the at least one processor is configured to execute the instructions to classify, based on the attribute information and the classification model, the observed feature values for training the respective stress estimation models.

4. The learning device according to claim 1,
wherein the at least one processor is configured to execute the instructions to classify, based on attribute information indicating attributes of the subjects, the observed feature values into the classes and then shuffle the observed feature values among the classes to increase the index for each of the classes.

5. The learning device according to claim 1,
wherein the at least one processor is configured to execute the instructions to classify the observed feature values into classes and then further subdivide a class, among the classes, in which the index is increased by subdividing the class.

6. The learning device according to claim 1,
wherein the at least one processor is configured to further execute the instructions to:
classify the observed feature values based on at least one of an observation target of the observed feature values and/or activity states of the subjects; and
select stress estimation feature values, which are feature values for stress estimation, from the observed feature values classified based on the classification,
wherein the at least one processor is configured to execute the instructions to train the stress estimation models for the respective classes, based on the stress estimation feature values and stress values which are correct answer corresponding to the stress estimation feature values.

7. The learning device according to claim 6,
wherein the at least one processor is configured to execute the instructions to select the stress estimation feature values based on a correlation between the observed feature values classified based on the classification and the stress values.

8. A stress estimation device comprising:
at least one memory configured to store instructions; and
at least one processor configured to execute the instructions to:
calculate classification scores representing confidence levels in which observed feature values of an estimation subject, who is a target of stress estimation, belong to respective classes;
acquire stress values of the estimation subject estimated based on the observed feature values by stress estimation models corresponding to the respective classes; and
calculate a stress estimate value obtained by integrating the stress values of the estimation subject estimated by the stress estimation models.

9. The stress estimation device according to claim 8,
wherein the at least one processor is configured to execute the instructions to calculate the classification scores based on classification model and attribute information indicating an attribute of the estimation subject, and
wherein the classification model is a model configured to classify observed feature values for training so that an index representing a correlation between the observed feature values and a stress value becomes higher than the correlation before the classification,
wherein the stress value is a correct answer corresponding to the observed feature values.

10. The stress estimation device according to claim 8,
wherein the at least one processor is configured to further execute the instructions to select stress estimation feature values, which are feature values for stress estimation, from the observed feature values,
wherein the at least one processor is configured to execute the instructions to calculate the stress estimate value obtained by integrating stress values of the estimation subject,
the stress values being estimated based on the stress estimation feature values by stress estimation models corresponding to the respective classes.

11. A learning method executed by a computer, the learning method comprising:
classifying observed feature values of subjects so that an index representing a correlation between the observed feature values and a stress value becomes higher than the correlation before the classification,
wherein the stress value is a correct answer corresponding to the observed feature values; and
training, based on the observed feature values and the stress value which is the correct answer, stress estimation models for respective classes divided at least by the classification,
wherein the stress estimation models each estimates a relation between the observed feature values and the stress value.

12.-14. (canceled)
